(19) United States
(12) Patent Application Publication
(10) Pub. No.: US 2014/0127698 A1
(43) Pub. Date: May 8, 2014

(54) REITERATIVE OLIGONUCLEOTIDE SYNTHESIS

(71) Applicant: LIFE TECHNOLOGIES CORPORATION, Carlsbad, CA (US)

(72) Inventor: Victoria SINGER, Eugene, OR (US)

(73) Assignee: LIFE TECHNOLOGIES CORPORATION, Carlsbad, CA (US)

(21) Appl. No.: 14/069,784

(22) Filed: Nov. 1, 2013

Related U.S. Application Data
(60) Provisional application No. 61/721,816, filed on Nov. 2, 2012.
(60) Provisional application No. 61/898,139, filed on Oct. 31, 2013.

(51) Int. Cl. C12Q 1/68

(52) U.S. Cl. CPC C12Q 1/686
USPC 435/6.12; 435/287.2

(57) ABSTRACT

In some embodiments, the disclosure relates generally to methods, as well as related compositions, systems, and kits, for nucleotide polymerization, oligonucleotide synthesis, detecting nucleotide polymerization, detecting the presence of a nucleic acid, oligonucleotide amplification and detection of oligonucleotide amplification, which can be conducted via an abortive transcription initiation reaction. In some embodiments, abortive transcription initiation reactions can generate multiple copies of an oligonucleotide which can be used to detect the presence of a nucleic acid or macromolecule. In some embodiments, generation of multiple copies of an oligonucleotide can be detected via a sensor that senses the presence of byproducts from a nucleotide incorporation or a nucleotide polymerization reaction. In some embodiments, the byproducts include pyrophosphate, hydrogen ion, charge transfer, and heat. In some embodiments, a abortive transcription initiation reaction can be conducted on a support that can be in contact with or capacitively coupled to at least one sensor. Optionally, the sensor comprises a field-effect transistor (FET).

This application claims the benefit of priority under 35 U.S.C. §119 to U.S. Provisional Application Nos. 61/721,816, filed Nov. 2, 2012, and 61/898,139, filed Oct. 31, 2013, the disclosures of all of which aforementioned applications are incorporated herein by reference in their entireties.

Throughout this application various publications, patents, and/or patent applications are referenced. The disclosures of these publications, patents, and/or patent applications in their entireties are hereby incorporated by reference into this application in order to more fully describe the state of the art to which this invention pertains.

### FIELD

Provided herein are methods, compositions, systems and kits for reiteratively synthesizing oligonucleotides on a sensor that detects the presence of byproducts from a nucleotide polymerization reaction.

### DRAWINGS

FIG. 1A is a schematic depicting a non-limiting embodiment of an abortive transcription initiation complex comprising a nucleic acid template (100) hybridized to an oligonucleotide probe (110). In some embodiments, the nucleic acid template (100) can be arranged in a 5′ to 3′ orientation (left to right, FIG. 1A, top) or in a 3′ to 5′ orientation (left to right, FIG. 1A, bottom). In some embodiments, an initiator (120) can hybridize to an abortive transcription initiation complex.

FIG. 1B is a schematic depicting a non-limiting embodiment of an abortive transcription initiation complex comprising a nucleic acid template (100) hybridized to an oligonucleotide probe (110). In some embodiments, the nucleic acid template can be attached to a linker molecule (130) which can be attached to a support (140). In some embodiments, an initiator (120) can hybridize to an abortive transcription initiation complex.

FIG. 2A is a schematic depicting a non-limiting embodiment of an abortive initiation cassette (150). In some embodiments, an abortive initiation cassette comprises a single nucleic acid strand having intramolecular hybridization regions. In some embodiments, the 5′ portion of a single-stranded nucleic acid can form the overhang portion of the abortive initiation cassette (FIG. 2A). In some embodiments, the 3′ portion of a single-stranded nucleic acid can form the overhang portion of the abortive initiation cassette.

FIG. 2B is a schematic depicting a non-limiting embodiment of a macromolecule (e.g., nucleic acid) (160) attached to an abortive initiation cassette (150). In some embodiments, an initiator (120) can hybridize to an abortive transcription initiation complex.

FIG. 2C is a schematic depicting a non-limiting embodiment of a macromolecule (e.g., nucleic acid) (160) attached to an abortive initiation cassette (150). In some embodiments, the macromolecule (160) can be attached to a linker molecule (130) which can be attached to a support (140). In some embodiments, an initiator (120) can hybridize to an abortive transcription initiation complex.

FIG. 3A is a schematic depicting a non-limiting embodiment of a macromolecule (e.g., polypeptide, lipid or sugar) (170) attached to an abortive initiation cassette (150). In some embodiments, an initiator (120) can hybridize to an abortive transcription initiation complex.

FIG. 3B is a schematic depicting a non-limiting embodiment of a target macromolecule (120) attached to an abortive initiation cassette (130), where the target macromolecule is attached to a linker (140) which is immobilized to a support (150). In some embodiments, the macromolecule (170) can be attached to a linker molecule (130) which can be attached to a support (140). In some embodiments, an initiator (120) can hybridize to an abortive transcription initiation complex.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the invention.

### DEFINITIONS

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

As utilized in accordance with exemplary embodiments provided herein, the following terms, unless otherwise indicated, shall be understood to have the following meanings:

As used herein the term “transcription” refers to an enzyme-catalyzed reaction that generates RNA having a nucleoside base sequence that is wholly or partially complementary to a nucleic acid template sequence. For example, a polymerase enzyme catalyzes nucleotide polymerization by forming phosphodiester bonds between nucleotides that successively bind a nucleic acid template. Generally, transcription includes several phases, including initiation, elongation and termination. During initiation, an initiator binds a nucleic acid template and a polymerase catalyzes phosphodiester bond formation between the initiator and a first nucleotide (polymerization). During elongation, a polymerase catalyzes phosphodiester bond formation between the first-polymerized nucleotide and subsequent nucleotides to generate an RNA transcript in a processive and template-dependent manner, without dissociation of the nascent transcript or the polymerase from the template. During termination, the polymerase dissociates from the nascent transcript as it reaches a termination sequence or reach the end of the template sequence

As used herein the term “abortive transcription initiation” or “abortive initiation” refers to an enzyme-catalyzed reaction in which an initiator binds a nucleic acid template and a polymerase catalyzes phosphodiester bond formation between the initiator and a first nucleotide. The polymerase continues to catalyze phosphodiester bond formation between the first-polymerized nucleotide and subsequent nucleotides to generate an RNA transcript in a processive and template-dependent manner, but the polymerase releases a short transcript (oligonucleotide approximately 2-50 nucleotides in length) before it reaches a termination sequence or before it reaches the end of the template sequence. The polymerase can catalyze multiple initiation transcript events to produce a plurality of short transcripts having substantially the same sequence as the first-released transcript. The polymerase reiteratively transcribes the same portion of the template and releases multiple copies of substantially the same nascent transcripts. The released short transcripts are called “abscripts” or “abortive initiation products”.

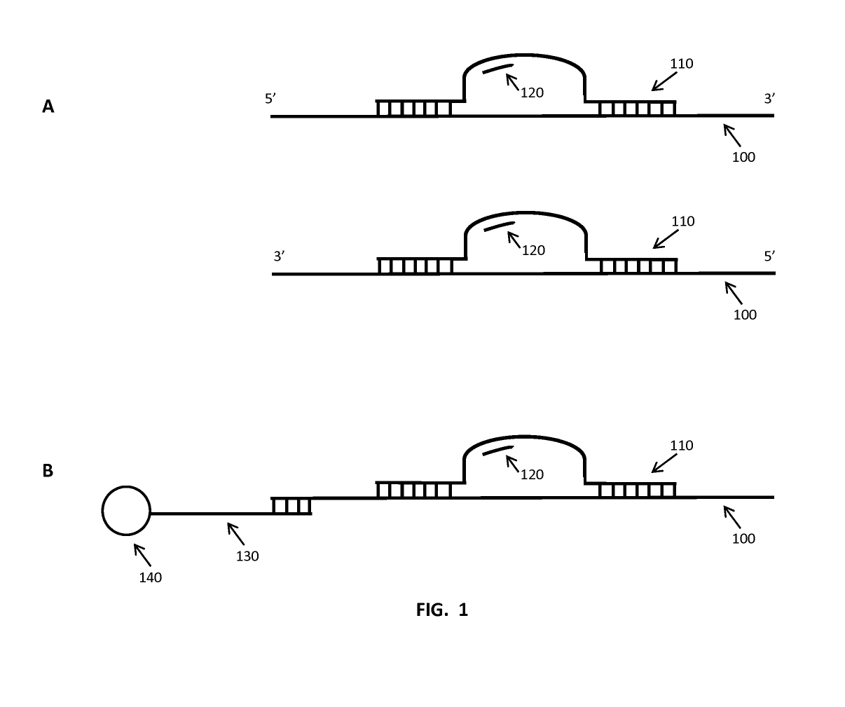

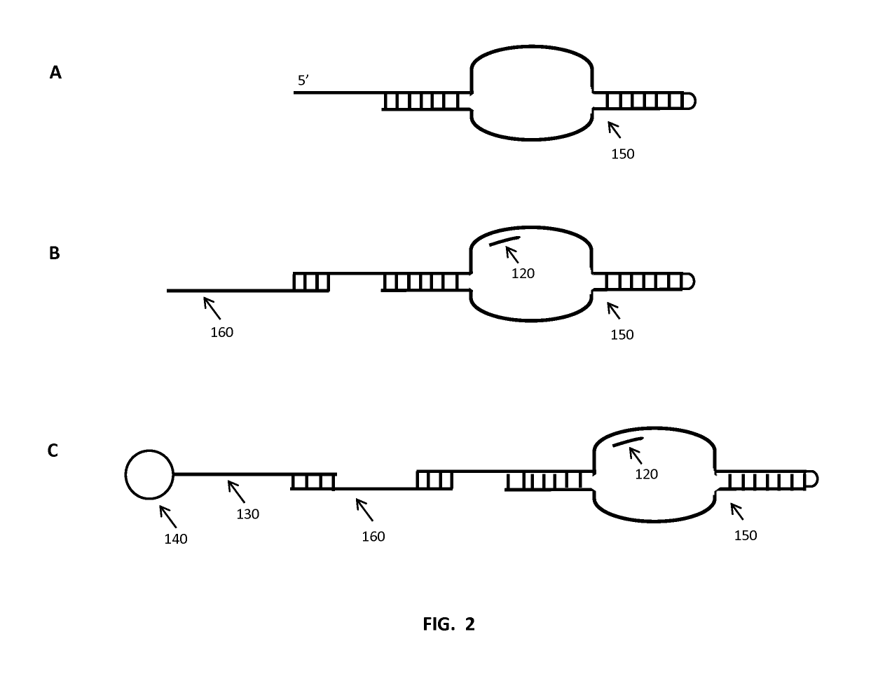

As used herein the term “transcription initiation bubble” refers to a nucleic acid-based structure having first and second duplex regions flanking a bubble region (FIGS. 1A and 2A). In some embodiments, the first and second duplex regions comprise wholly or partially complementary double stranded regions. In some embodiments, the bubble region comprises two nucleic acid strands that do not form a nucleic acid duplex. In some embodiments, the transcription initiation bubble comprises two or more nucleic acid strands having hybridization regions to form the flanking first and second duplex regions and the intervening bubble region (FIG. 1A). In some embodiments, the transcription initiation bubble comprises a single nucleic acid strand having intramolecular hybridization regions to form the flanking first and second duplex regions and the intervening bubble region (FIG. 2A, e.g., an abortive initiation cassette).

As used herein the term “abortive transcription initiation reaction” refers to a reaction comprising reagents for producing abortive initiation products. In some embodiments, an abortive transcription initiation reaction comprises a transcription initiation bubble. Optionally, the abortive transcription initiation reaction also includes any one or any combination of: (i) a transcription initiator, (ii) one or more nucleotides, and/or (iii) at least one polymerase.

As used herein the term “initiator” or “transcription initiator” refers to a mononucleoside, mononucleotide or oligonucleotide (e.g., having two or more nucleosides), or analog thereof, which can serve as an RNA transcription primer. In some embodiments, an initiator can hybridize to a transcription initiation bubble (FIG. 1A or 2A).

As used herein the term “binding partner(s)” refers to two molecules, or portions thereof, which have a specific binding affinity for one another and typically will bind to each other in preference to binding to other molecules. Typically, binding partners can be polypeptides that can bind or associate with each other. Interactions between the binding partners can be strong enough to allow enrichment and/or purification of a conjugate that comprises a binding partner and a molecule associated with it (e.g., a biotinylated abortive initiation cassette). An example of commonly used binding partners includes biotin and streptavidin. Other examples include: biotin or desthiobiotin or photoactivatable biotin and their binding partners avidin, streptavidin, Neutravidin™, or Captavidin™. Another binding partner for biotin can be a biotin-binding protein from chicken (Hytonen, et al., BMC Structural Biology 7:8). Other examples of molecules that function as binding partners include: His-tags which bind with nickel, cobalt or copper; Ni-NTA which binds cysteine, histidine, or histidine patch; maltose which binds with maltose binding protein (MBP); lectin-carbohydrate binding partners; calcium-calcium binding protein (CBP); acetylcholine and receptor-acetylcholine; protein A and anti-FLAG antibody; GST and glutathione; uracil DNA glycosylase (UDG) and ugi (uracil-DNA glycosylase inhibitor) protein; antigen or epitope tags which bind to antibody or antibody fragments, particularly antigens such as digoxigenin, fluorescein, dinitrophenol or bromodeoxyuridine and their respective antibodies; mouse immunoglobulin and goat anti-mouse immunoglobulin; IgG bound and protein A; receptor-receptor agonist or receptor antagonist; enzyme-enzyme cofactors; enzyme-enzyme inhibitors; and thyroxine-cortisol.

As used herein the term “nucleotides” refers to any compound that can bind selectively to, or can be polymerized by, a polymerase. In some embodiments, a nucleotide can be a naturally-occurring nucleotide, or analog thereof. In some embodiments, a nucleotide comprises a base, sugar and phosphate moieties. In some embodiments, a nucleotide can lack a base, sugar or phosphate moiety. In some embodiments, a nucleotide comprises a base including: cytosine, thymine, adenine, guanine, hypoxanthine, uracil, or analogs thereof. In some embodiments, a nucleotide can include a chain of phosphorus atoms comprising three, four, five, six, seven, eight, nine, ten or more phosphorus atoms. In some embodiments, a phosphorus chain can be attached to any carbon of a sugar ring, such as the 5′ carbon. In some embodiments, a phosphorus chain can be linked to the sugar with an intervening O or S. In some embodiments, one or more phosphorus atoms in a phosphorus chain can be part of a phosphate group having P and O. In some embodiments, the phosphorus atoms in the chain can be linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In some embodiments, the phosphorus atoms in the chain can have a side group having O, BH₃, or S. In some embodiments, a phosphorus atom having a side group other than O can be a substituted phosphate group. In some embodiments, a nucleotide can be attached to a label (e.g., reporter moiety). In some embodiments, a label can be a fluorophore. In some embodiments, a fluorophore can be attached to the terminal phosphate group (or substitute phosphate group). In some embodiments, a nucleotide can comprise a non-oxygen moiety (e.g., thio- or borano-moieties) that replaces an oxygen moiety that bridges the alpha phosphate and the sugar of the nucleotide, or bridges the alpha and beta phosphates of the nucleotide, or bridges the beta and gamma phosphates of the nucleotide, or between any other two phosphates of the nucleotide, or any combination thereof. In some embodiments, nucleotides can be biotinylated. In some embodiments, a nucleotide can be a ribonucleotide, deoxyribonucleotide, ribonucleotide polyphosphate, deoxyribonucleotide polyphosphate, peptide nucleotides, metallonucleosides, phosphonate nucleosides, and modified phosphate-sugar backbone nucleotides, analogs, derivatives, variants or modified versions thereof.

As used herein the term “terminator nucleotide” and “chain terminating nucleotide” refers to a nucleotide or nucleotide analog that can be incorporated by a polymerase into a nascent nucleic acid chain, but prevents polymerization of a subsequent nucleotide. In some embodiments, a terminator nucleotide comprises a blocking moiety joined to any position of a base, sugar or phosphate group, where the blocking moiety prevents polymerization of a subsequent nucleotide. In some embodiments, a blocking moiety includes small and large blocking moieties. In some embodiments, a terminator nucleotide includes a blocking moiety at a 2′ or 3′ sugar position. For example, a terminator nucleotide includes a dideoxynucleotide comprising an —H at a 3′ sugar position. In some embodiments, a blocking moiety includes any compound: amine, alkyl, alkenyl, alkynyl, alkyl amide, aryl, ether, ester, benzyl, propargyl, propynyl, phosphate, or analog thereof. For example, a blocking moiety can be a 3′-O-allyl moiety (Ruparel, et al., 2005 Proc. Natl. Acad. Sci. USA 102:5932-5937). In some embodiments, a blocking moiety includes: fluorenylmethyloxycarbonyl (FMOC), 4-(anisyl)diphenylmethyltrityl (MMTr), dimethoxytrityl (DMTr), monomethoxytrityl, trityl (Tr), benzoyl (Bz), isobutyryl (ib), pixyl (pi), ter-butyl-dimethylsilyl (TBMS), and 1-(2-fluorophenyl)-4-methoxypiperidin 4-yl (FPMP). See also T W Greene 1981, in “Protective Groups in Organic Synthesis”, publishers Wiley-Interscience; Beaucage and Iyer 1992 Tetrahedron, 48:2223-2311; Beaucage and Iyer 1993 Tetrahedron 49:10441-10488; and Scaringe et al., 1998 J. Am. Chem. Soc. 120:11820-11821. In some embodiments, a blocking moiety includes a fluorophore.

As used herein the term “elongation nucleotide” refers to a nucleotide or nucleotide analog that can be incorporated by a polymerase into a nascent nucleic acid chain, but does not prevent polymerization of a subsequent nucleotide.

As used herein the terms “detectable reporter moiety” and “reporter moiety” refer to a compound that generates, or causes to generate, a detectable signal. In some embodiments, a detectable reporter moiety can be detected as: luminescence, photoluminescence, electroluminescence, bioluminescence, chemiluminescence, fluorescence, phosphorescence, colorimetric, radio activity, electrochemical, mass spectrometry, Raman, hapten, affinity tag, atom, or an enzymatic activity. In some embodiments, a detectable reporter moiety can generate a detectable signal resulting from a chemical or physical change (e.g., heat, light, electrical, pH, salt concentration, enzymatic activity, or proximity events). A proximity event can include two detectable reporter moieties approaching each other, or associating with each other, or binding each other. In some embodiments, a fluorescent moiety includes: rhodols; resorufins; coumarins; xanthenes; acridines; fluoresceins; rhodamines; erythrins; cyanins; phthalaldehydes; naphthylamines; fluorescamines; benzoxadiazoles; stilbenes; pyrenes; indoles; borapolyazaindacenes; quinazolinones; eosin; erythrosin; Malachite green; CY dyes (GE Biosciences), including Cy3 (and its derivatives) and Cy5 (and its derivatives).

### DESCRIPTION

In some embodiments, the disclosure relates generally to methods, as well as related compositions, systems, and kits, for nucleotide polymerization, nucleotide incorporation, oligonucleotide synthesis, detecting nucleotide polymerization, detecting the presence of a nucleic acid, oligonucleotide amplification and detection of oligonucleotide amplification. In some embodiments, multiple rounds of nucleotide polymerization can generate a plurality of oligonucleotides. In some embodiments, nucleotide polymerization can be conducted reiteratively. In some embodiments, nucleotide polymerization can be conducted via a transcription-based reaction. In some embodiments, nucleotide polymerization can be conducted via an abortive transcription initiation reaction. In some embodiments, abortive transcription initiation reactions can generate multiple copies of an oligonucleotide (amplification) which can be used to detect the presence of a nucleic acid or macromolecule. In some embodiments, generation of multiple copies of an oligonucleotide can be detected (e.g., signal amplification) via mass spectrophotometry, capillary electrophoresis, fluorescence or rapid TLC. In some embodiments, generation of multiple copies of an oligonucleotide can generate byproducts of a nucleotide incorporation reaction or of a nucleotide polymerization reaction. In some embodiments, a nucleotide incorporation reaction or a nucleotide polymerization reaction can be located at or near a sensor that senses the presence of byproducts from the reaction. In some embodiments, the byproducts include pyrophosphate, hydrogen ion, charge transfer, and heat. In some embodiments, a nucleotide incorporation reaction or a nucleotide polymerization reaction can be conducted on a support, for example on a surface or the support that can be in contact with or capacitively coupled to the sensor. In some embodiments, a nucleotide incorporation reaction or a nucleotide polymerization reaction can be conducted on a support, for example a surface that can be in contact with or capacitively coupled to at least one field-effect transistor (FET).

In some embodiments, the disclosure relates generally to methods (as well as related compositions, systems, and kits) for nucleotide polymerization, nucleotide incorporation, oligonucleotide synthesis, detecting nucleotide polymerization, detecting the presence of a nucleic acid, oligonucleotide amplification and detection of oligonucleotide amplification, the method comprising: conducting an abortive transcription initiation reaction on a support that is capacitively coupled to one or more sensors that detect a nucleotide polymerization reaction byproduct. In some embodiments, the support comprises a surface having, for example, an outer or top-most layer or boundary of an object. In some embodiments, the methods comprise: conducting an abortive transcription initiation reaction on a surface that is in contact with or capacitively coupled to one or more sensors that detect a nucleotide polymerization reaction byproduct. In some embodiments, the sensor comprises a field-effect transistor (FET). In some embodiments, the abortive transcription initiation reaction includes a nucleic acid template (100) hybridized to an oligonucleotide probe (110) to form a transcription initiation bubble structure (FIGS. 1A and B), an initiator (120), one or more nucleotides, and at least one polymerase, so as to polymerize the one or more nucleotides onto the initiator; and detecting the nucleotide polymerization by a change in an electrical parameter at the sensor. In some embodiments, the polymerization of one or more nucleotides onto the initiator synthesizes an oligonucleotide.

In some embodiments, methods for nucleotide polymerization comprise: conducting an abortive transcription initiation reaction on a surface that is in contact with or capacitively coupled to at least one sensor, wherein the abortive transcription initiation reaction includes an abortive initiation cassette (AIC) (150) (where the abortive initiation cassette forms a transcription initiation bubble structure, FIGS. 2A, B and C), an initiator (120), one or more nucleotides, and at least one polymerase, so as to polymerize the one or more nucleotides onto the initiator; and detecting the nucleotide polymerization by a change in an electrical parameter at the sensor. Optionally, the sensor comprises a field-effect transistor (FET).

In some embodiments, methods for nucleotide polymerization comprise: conducting an abortive transcription initiation reaction on a surface that is in contact with or capacitively coupled to at least one sensor, wherein the abortive transcription initiation reaction includes a macromolecule (160) or (170) joined to an abortive initiation cassette (AIC) (150) (where the abortive initiation cassette forms a transcription initiation bubble structure, FIGS. 2A-C and 3A-B), an initiator (120), one or more nucleotides, and at least one polymerase, so as to polymerize the one or more nucleotides onto the initiator; and detecting the nucleotide polymerization by a change in an electrical parameter at the sensor. In some embodiments, the abortive initiation cassette comprises a single nucleic acid strand having intramolecular hybridization regions that form the transcription initiation bubble (FIG. 2A), or comprises two or more nucleic acid strands having hybridization regions that form the transcription initiation bubble (FIG. 1A). In some embodiments, macromolecules include nucleic acids, polypeptides, lipids and sugars. In some embodiments, the polymerization of one or more nucleotide onto the initiator synthesizes an oligonucleotide. Optionally, the sensor comprises a field-effect transistor (FET).

In some embodiments, methods for synthesizing oligonucleotides comprise conducting an abortive transcript initiation reaction at or near a sensor that detects or senses the presence of byproducts from a nucleotide incorporation reaction or a nucleotide polymerization reaction. In some embodiments, the byproducts include pyrophosphate, hydrogen ion, charge transfer, and heat. In some embodiments, the sensor comprises a field-effect transistor (FET). In some embodiments, the byproducts are detectable by the sensor.

In some embodiments, methods for synthesizing oligonucleotides comprise conducting an abortive transcript initiation reaction with one or more nucleotides, including for example, a plurality of nucleotides, or multiple nucleotides or one type or more than one type. In some embodiments, the plurality of nucleotides includes at least one type of terminator nucleotides, or at least one type of elongation nucleotide, or a mixture of terminator and elongation nucleotides.

In some embodiments, methods for synthesizing oligonucleotides comprise: providing a nucleic acid template hybridized to an oligonucleotide probe to form a transcription initiation bubble structure; contacting the transcription initiation bubble structure with an initiator, a polymerase and one or more nucleotides, under conditions suitable for nucleotide polymerization to synthesize a plurality of abortive transcript products having sequences that can be complementary to at least a portion of the bubble structure, wherein the transcription initiation bubble structure can be located at or near a sensor that senses the presence of byproducts from the nucleotide polymerization reaction. In some embodiments, the oligonucleotide synthesis produces byproducts comprising pyrophosphate, hydrogen ion, charge transfer, or heat. In some embodiments, byproducts of oligonucleotide synthesis are detectable by the sensor. In some embodiments, the sensor comprises a field-effect transistor (FET). In some embodiments, the nucleotides comprise at least one terminator nucleotide and/or at least one elongation nucleotide. In some embodiments, the polymerase comprises an RNA polymerase.

In some embodiments, methods for synthesizing oligonucleotides comprise: hybridizing a nucleic acid template (100) to an oligonucleotide probe (110) to form a transcription initiation bubble structure; contacting the transcription initiation bubble structure with an initiator (120), a polymerase and one or more nucleotides, under conditions suitable for nucleotide polymerization to synthesize a plurality of abortive transcript products having sequences that can be complementary to at least a portion of the bubble structure (FIGS. 1-3), wherein the transcription initiation bubble structure can be located at or near a sensor that senses the presence of byproducts from the nucleotide polymerization reaction. In some embodiments, the oligonucleotide synthesis produces byproducts comprising pyrophosphate, hydrogen ion, charge transfer, or heat. In some embodiments, byproducts of oligonucleotide synthesis are detectable by the sensor. In some embodiments, the sensor comprises a field-effect transistor (FET). In some embodiments, the nucleotides comprise at least one terminator nucleotide and/or at least one elongation nucleotide. In some embodiments, the polymerase comprises an RNA polymerase.

In some embodiments, methods for synthesizing oligonucleotides comprise: providing a macromolecule (160) or (170) joined to an abortive initiation cassette (AIC) (150) having a transcription initiation bubble structure; contacting the transcription initiation bubble structure with an initiator (120), a polymerase and one or more nucleotides, under conditions suitable for nucleotide polymerization to synthesize a plurality of abortive transcript products having sequences that can be complementary to at least a portion of the bubble structure (FIGS. 2A-C), wherein the transcription initiation bubble structure can be located at or near a sensor that senses the presence of byproducts from the nucleotide polymerization reaction. In some embodiments, the oligonucleotide synthesis produces byproducts comprising pyrophosphate, hydrogen ion, charge transfer, or heat. In some embodiments, byproducts of oligonucleotide synthesis are detectable by the sensor. In some embodiments, the sensor comprises a field-effect transistor (FET). In some embodiments, the nucleotides comprise at least one terminator nucleotide and/or at least one elongation nucleotide. In some embodiments, the polymerase comprises an RNA polymerase.

In some embodiments, methods for synthesizing oligonucleotides comprise: providing a transcription initiation site at or near a sensor that senses the presence of byproducts from a nucleotide incorporation reaction, wherein the a transcription initiation site comprises a nucleic acid template (100) hybridized with an oligonucleotide probe (110) that forms a transcription initiation bubble structure; and contacting the transcription initiation bubble structure with (i) a nucleic acid initiator (120) and (ii) a polymerase and (iii) a terminator nucleotide under conditions suitable for the polymerase to reiteratively synthesize oligonucleotides, wherein the initiator hybridizes to the transcription initiation bubble structure (FIGS. 1-3). In some embodiments, the oligonucleotide synthesis produces byproducts comprising pyrophosphate, hydrogen ion, charge transfer, or heat. In some embodiments, byproducts of oligonucleotide synthesis are detectable by the sensor. In some embodiments, the sensor comprises a field-effect transistor (FET). In some embodiments, the nucleotides comprise at least one terminator nucleotide and/or at least one elongation nucleotide. In some embodiments, the polymerase comprises an RNA polymerase.

In some embodiments, methods for synthesizing oligonucleotides comprise: providing a transcription initiation site at or near a sensor that senses the presence of byproducts from a nucleotide incorporation reaction, wherein the a transcription initiation site comprises a nucleic acid template (100) hybridized with an oligonucleotide probe (110) that forms a transcription initiation bubble structure; and contacting the transcription initiation bubble structure with (i) a nucleic acid initiator (120) and (ii) a polymerase and (iii) one or more elongation nucleotides under conditions suitable for the polymerase to reiteratively synthesize oligonucleotides, wherein the initiator hybridizes to the transcription initiation bubble structure (FIGS. 1-3). In some embodiments, the oligonucleotide synthesis produces byproducts comprising pyrophosphate, hydrogen ion, charge transfer, or heat. In some embodiments, byproducts of oligonucleotide synthesis are detectable by the sensor. In some embodiments, the sensor comprises a field-effect transistor (FET). In some embodiments, the nucleotides comprise at least one terminator nucleotide and/or at least one elongation nucleotide. In some embodiments, the polymerase comprises an RNA polymerase.

In some embodiments, methods for synthesizing oligonucleotides comprise: providing a transcription initiation site on a sensor that senses the presence of byproducts from a nucleotide incorporation reaction, wherein the a transcription initiation site comprises a nucleic acid template (100) hybridized with an oligonucleotide probe (110) that forms a transcription initiation bubble structure; and contacting the transcription initiation bubble structure with (i) a nucleic acid initiator (120) and (ii) a polymerase and (iii) at least one terminator nucleotide and (iv) at least one elongation nucleotides, under conditions suitable for the polymerase to reiteratively synthesize oligonucleotides, wherein the initiator hybridizes to the transcription initiation bubble structure (FIGS. 1-3). In some embodiments, the oligonucleotide synthesis produces byproducts comprising pyrophosphate, hydrogen ion, charge transfer, or heat. In some embodiments, byproducts of oligonucleotide synthesis are detectable by the sensor. In some embodiments, the sensor comprises a field-effect transistor (FET). In some embodiments, the nucleotides comprise at least one terminator nucleotide and/or at least one elongation nucleotide. In some embodiments, the polymerase comprises an RNA polymerase.

In some embodiments, methods for synthesizing oligonucleotides comprise: providing a transcription initiation site on a sensor that senses the presence of byproducts from a nucleotide incorporation reaction, wherein the a transcription initiation site comprises a target macromolecule (160) or (170) attached to an Abortive Initiation Cassette (AIC) (150); and contacting the transcription initiation site with (i) a nucleic acid initiator (120) and (ii) a polymerase and (iii) at least one terminator nucleotide under conditions suitable for the polymerase to reiteratively synthesize oligonucleotides, wherein the initiator hybridizes to the transcription initiation bubble structure (FIGS. 2A, B). In some embodiments, the Abortive Initiation Cassette forms a transcription initiation bubble structure. In some embodiments, the macromolecule comprises a nucleic acid, polypeptide, lipid or sugar. In some embodiments, the oligonucleotide synthesis produces byproducts comprising pyrophosphate, hydrogen ion, charge transfer, or heat. In some embodiments, byproducts of oligonucleotide synthesis are detectable by the sensor. In some embodiments, the sensor comprises a field-effect transistor (FET). In some embodiments, the nucleotides comprise at least one terminator nucleotide and/or at least one elongation nucleotide. In some embodiments, the polymerase comprises an RNA polymerase.

In some embodiments, methods for synthesizing oligonucleotides comprise: providing a transcription initiation site on a sensor that senses the presence of byproducts from a nucleotide incorporation reaction, wherein the a transcription initiation site comprises a target macromolecule (160) or (170) attached to an Abortive Initiation Cassette (AIC) (150); and contacting the transcription initiation site with (i) a nucleic acid initiator (120) and (ii) a polymerase and (iii) at least one elongation nucleotides under conditions suitable for the polymerase to reiteratively synthesize oligonucleotides, wherein the initiator hybridizes to the transcription initiation bubble structure. In some embodiments, the Abortive Initiation Cassette forms a transcription initiation bubble structure (FIGS. 2A, B). In some embodiments, the macromolecule comprises a nucleic acid, polypeptide, lipid or sugar. In some embodiments, the oligonucleotide synthesis produces byproducts comprising pyrophosphate, hydrogen ion, charge transfer, or heat. In some embodiments, byproducts of oligonucleotide synthesis are detectable by the sensor. In some embodiments, the sensor comprises a field-effect transistor (FET). In some embodiments, the nucleotides comprise at least one terminator nucleotide and/or at least one elongation nucleotide. In some embodiments, the polymerase comprises an RNA polymerase.

In some embodiments, methods for synthesizing oligonucleotides comprise: providing a transcription initiation site on a sensor that senses the presence of byproducts from a nucleotide incorporation reaction, wherein the a transcription initiation site comprises a target macromolecule (160) or (170) attached to an Abortive Initiation Cassette (AIC) (150); and contacting the transcription initiation site with (i) a nucleic acid initiator (120) and (ii) a polymerase and (iii) at least one terminator nucleotide and (iv) at least one elongation nucleotides under conditions suitable for the polymerase to reiteratively synthesize oligonucleotides, wherein the initiator hybridizes to the transcription initiation bubble structure. In some embodiments, the Abortive Initiation Cassette forms a transcription initiation bubble structure. In some embodiments, the macromolecule comprises a nucleic acid, polypeptide, lipid or sugar. In some embodiments, the oligonucleotide synthesis produces byproducts comprising pyrophosphate, hydrogen ion, charge transfer, or heat. In some embodiments, byproducts of oligonucleotide synthesis are detectable by the sensor. In some embodiments, the sensor comprises a field-effect transistor (FET). In some embodiments, the nucleotides comprise at least one terminator nucleotide and/or at least one elongation nucleotide. In some embodiments, the polymerase comprises an RNA polymerase.

In some embodiments, oligonucleotides can be reiteratively synthesized by contacting an initiator (e.g., a mononucleoside or mononucleotide) with a terminator nucleotide to generate a di-nucleotide product.

In some embodiments, oligonucleotides can be reiteratively synthesized by contacting an initiator (e.g., a di-nonucleotide) with a terminator nucleotide to generate a tri-nucleotide product.

In some embodiments, oligonucleotides can be reiteratively synthesized by contacting an initiator (e.g., a tri-nonucleotide) with a terminator nucleotide to generate a tetra-nucleotide product.

In some embodiments, oligonucleotides can be reiteratively synthesized by contacting an initiator (e.g., a mononucleoside or mononucleotide) with a terminator nucleotide and one or more elongation nucleotides to generate an oligonucleotide product having two or more nucleotides in length.

In some embodiments, oligonucleotides can be reiteratively synthesized by contacting an initiator (e.g., a di-nucleotide) with a terminator nucleotide and one or more elongation nucleotides can generate an oligonucleotide product having three or more nucleotides in length.

In some embodiments, oligonucleotides can be reiteratively synthesized by contacting an initiator (e.g., a tri-nucleotide) with a terminator nucleotide and one or more elongation nucleotides can generate an oligonucleotide product having four or more nucleotides in length.

In some embodiments, oligonucleotides can be reiteratively synthesized by contacting an initiator (e.g., a mononucleoside or mononucleotide) with one or more elongation nucleotides can generate an oligonucleotide product having two or more nucleotides in length.

In some embodiments, oligonucleotides can be reiteratively synthesized by contacting an initiator (e.g., a di-nucleotide) with one or more elongation nucleotides can generate an oligonucleotide product having three or more nucleotides in length.

In some embodiments, oligonucleotides can be reiteratively synthesized by contacting an initiator (e.g., a tri-nucleotide) with one or more elongation nucleotide can generate an oligonucleotide product having four or more nucleotides in length.

In some embodiments, an oligonucleotide synthesis reaction can be coupled to an amplification reaction (e.g., PCR or loop-mediated isothermal amplification (Notomi 2000 Nucleic Acids Research 28:e63)) to further amplify the number of oligonucleotides synthesized.

In some embodiments, a multiplex oligonucleotide synthesis reaction comprises: providing a nucleic acid sample (which contains a polynucleotide of interest) on or near a sensor that senses the presence of byproducts from a nucleotide incorporation reaction; contacting the nucleic acid sample with a mixture of oligonucleotide probes or a mixture of abortive initiation cassettes so as to bind the polynucleotide of interest to the oligonucleotide probe or to the abortive initiation cassette; contacting the bound polynucleotide of interest with an initiator (e.g., di-nucleotide), a polymerase (e.g., RNA polymerase), and at least one nucleotide under conditions suitable for abortive transcription initiation; and detecting byproducts from the abortive transcription initiation reaction.

In some embodiments, abortive transcription initiation can be used to detect the presence of a polynucleotide of interest. For example abortive transcription initiation can be used in combination with amplification (e.g., PCR) or sequencing or other nucleic acid reactions. In some embodiments, products of an amplification or sequencing reaction can be detected by hybridizing the products with an oligonucleotide probe having one or more regions that hybridize with at least a portion of the amplification or sequencing product to form a transcription initiation bubble structure. In some embodiments, products of an amplification or sequencing reaction can be detected by hybridizing the products with an abortive initiation cassette comprising a terminal portion (5′ or 3′ end) having a capture sequence to detect the presence of a PCR or sequencing product. In some embodiments, the 5′ or 3′ terminal end of the abortive initiation cassette includes a region that hybridizes with at least a portion of the amplification or sequencing product for capturing the product. The abortive transcription initiation reaction can be conducted on or near a sensor that senses the presence of byproducts from a nucleotide incorporation reaction, and include an initiator, a polymerase (e.g., RNA polymerase), and at least one nucleotide under conditions suitable for abortive transcription initiation.

In some embodiments, abortive transcription initiation can be used to detect the presence of RNA, including for example, polyA-RNA. In some embodiments, RNA can be hybridized with an abortive transcription initiation complex (FIG. 1A), or an abortive initiation cassette (FIGS. 2A and B), where the abortive transcription initiation complex includes a region that can hybridize with a portion of the RNA. For example, a polyA portion of an RNA molecule can hybridize with the 5′ end of an abortive transcription initiation complex, or the 5′ end of an abortive initiation cassette, having a sequence capable of hybridizing with the polyA portion. In some embodiments, the 5′ end of the abortive transcription initiation complex, or the abortive initiation cassette, comprises a polyT or polyU sequence. In some embodiments, any portion of the abortive transcription initiation complex, or the abortive initiation cassette, comprises any sequence that can hybridize with any portion of the RNA.

In some embodiments, abortive transcription initiation can be used for signal amplification. For example, a DNA polymerization reaction can be conducted with an abortive initiation cassette, an initiator (e.g., a specific di-nucleotide), a polymerase (e.g., RNA polymerase), and at least one nucleotide under conditions suitable for abortive transcription initiation. The abortive initiation cassette can hybridize to a desired product of DNA polymerization and permit abortive transcription initiation. The reaction can be conducted on or near a sensor that senses the presence of byproducts from a nucleotide incorporation reaction.

In some embodiments, abortive transcription initiation can be used to detect a binding event. For example, abortive transcription initiation can be coupled with Project Flux Capacitor or acid generators to detect binding events. For example, an abortive initiation cassette can be attached to an antibody and the antibody can be bound to its cognate antigen. In a multiplex format, a mixture of different unique abortive initiation cassettes (e.g., having different sequences) can be attached to different antibodies. Alternatively, a mixture of different unique abortive initiation cassettes (e.g., having different sequences) can be attached to one member of a binding partner (e.g., streptavidin/biotin, lectin/carbohydrate(sugar), cell surface receptor/ligand, substrate/enzyme (or inhibitor), virus/target cell, and the like).

In some embodiments, the disclosure relates generally to methods (as well as related compositions, systems, and kits) for nucleotide polymerization, nucleotide incorporation, oligonucleotide synthesis, detecting nucleotide polymerization, detecting the presence of a nucleic acid, oligonucleotide amplification and detection of oligonucleotide amplification, wherein conditions suitable for synthesizing oligonucleotides comprise any condition suitable for conducting a transcription reaction or an abortive transcription initiation reaction. For example, conditions suitable for synthesizing oligonucleotides include well known parameters, such as: time, temperature, pH, buffers, reagents, cations, salts, co-factors, nucleotides, nucleic acids, and enzymes. In some embodiments, a buffer can include Tris, Tricine, HEPES, MOPS, ACES, MES, or inorganic buffers such as phosphate or acetate-based buffers which can provide a pH range of about 4-12. In some embodiments, a buffer can include chelating agents such as EDTA or EGTA. In some embodiments, a buffer can include dithiothreitol (DTT), glycerol, spermidine, and/or BSA (bovine serum albumin).

In some embodiments, suitable conditions include monovalent ions, divalent cations and/or a reducing agent. In some embodiments, a monovalent ion includes KCl, K-acetate, NH₄-acetate, K-glutamate, NH₄Cl, and ammonium sulfate. In some embodiments, a divalent cation includes calcium, magnesium, manganese, zinc, and cobalt. For example, sources of magnesium can include MgCl₂ and Mg-acetate. In some embodiments, suitable conditions include MgCl₂ at about 0.1-10 mM range, or about 0.5-5 mM range, or about 0.5-2 mM range.

In some embodiments, suitable conditions include a high salt concentration, including about 100 mM, or employing alternative monovalent cations such as K⁺ or Na+ or Rb+. In some embodiments, suitable conditions include sulfhydral reducing agents including 2-mercaptoethanol (e.g., at about 1-3 mM) or 5,5′-dithio-bis-(2-nitrobenoic) acid. In some embodiments, suitable conditions include a high molar ration of polymerase enzyme (e.g., RNA polymerase) to nucleic acid template. In some embodiments, suitable conditions include mutant enzymes (e.g., RNA polymerases) that exhibit elevated rates of abortive transcription initiation. For example, E. coli RNA polymerase comprising arginine at position 529 substituted with cysteine can perform elevated rates of abortive initiation. In some embodiments, suitable conditions include use of promoter sequences that generate elevated rates of abortive initiation (e.g., galP2 promoter).

In some embodiments, suitable conditions include conducting oligonucleotide synthesis in aqueous reaction conditions. In some embodiments, suitable conditions include conducting oligonucleotide synthesis in a tube, a well, an oil-and-water emulsion droplet or an agarose droplet (Yang 2010 Lab Chip 10(21):2841-2843).

In some embodiments, suitable conditions include isothermal or cycling temperature conditions. In some embodiments, suitable conditions include temperature ranges of about 22-100° C., or about 25-85° C., or about 25-75° C., or about 25-55° C.

In some embodiments, suitable conditions include conducting an abortive initiation reaction with single-stranded nucleic acid templates. For example, a double-stranded nucleic template can be denatured using heat (e.g., 65-100° C.) and/or NaOH (e.g., about 0.1-2 N NaOH).

In some embodiments, suitable conditions include hybridizing an oligonucleotide probe with a nucleic acid template to form a bubble structure (e.g., transcription initiation bubble structure). For example, hybridization can be conducted at about 20-85° C., or about 25-70° C., or about 35-50° C.

In some embodiments, suitable conditions include hybridizing an oligonucleotide probe with a nucleic acid template to form a bubble structure for less than 5 minutes, or about 5-120 minutes, or about 5-60 minutes, or about 5-30 minutes.

In some embodiments, conditions suitable for synthesizing oligonucleotides comprise any combination and in any order: forming a transcription initiation bubble structure; contacting a transcription initiation bubble structure with an initiator; contacting a transcription initiation bubble structure with at least one polymerase enzyme; contacting a transcription initiation bubble structure with at least one nucleotide (elongation nucleotides and/or terminator nucleotides); and/or conducting oligonucleotide synthesis (e.g., abortive transcription initiation). Suitable conditions include adding components of an abortive initiation reaction simultaneously (or essentially simultaneously), or adding components separately. For example, components include but are not limited to: nucleic acid templates or macromolecules; oligonucleotide probes or abortive initiation cassettes; initiators; polymerase enzymes; terminator nucleotides; and/or elongation nucleotides. In some embodiments, forming a transcription initiation bubble structure comprises hybridizing a nucleic acid template with an oligonucleotide probe to form a nucleic acid structure having a first duplex region, a single-stranded bubble structure and a second duplex region (FIGS. 1A and B). In some embodiments, an abortive initiation cassette can form a transcription initiation bubble structure comprising a first duplex region, a single-stranded bubble structure and a second duplex region (FIG. 2A). In some embodiments, an abortive initiation cassette can be joined or attached to at least one macromolecule (FIGS. 2B and C, 3A and B).

In some embodiments, synthesizing oligonucleotides can be conducted in any type of reaction vessel. For example, a reaction vessel includes any type of tube, column or well (e.g., 96-well plate). In some embodiments, synthesizing oligonucleotides can be practiced in any type of thermal-control apparatus. In some embodiments, a thermal-control apparatus can maintain a desired temperature, or can elevate and decrease the temperature, or can elevate and decrease the temperature for multiple cycles. In some embodiments, a thermal-control apparatus can maintain a temperature range of about 0° C.-100° C., or can cycle between different temperature ranges of about 0° C.-100° C. Examples of thermal-control apparatus include: a water bath and thermal cycler machine. Many thermal cycler machines are commercially-available, including (but not limited to) Applied Biosystems, Agilent, Eppendorf, Bio-Rad and Bibby Scientific.

In some embodiments, the disclosure relates generally to methods (as well as related compositions, systems, and kits) for nucleotide polymerization, nucleotide incorporation, oligonucleotide synthesis, detecting nucleotide polymerization, detecting the presence of a nucleic acid, oligonucleotide amplification and detection of oligonucleotide amplification, wherein the method can be conducted with nucleic acid templates. In some embodiments, a nucleic acid template (100) comprises a single-stranded nucleic acid template, or a denatured double-strand nucleic acid template to form a single-stranded template. In some embodiments, nucleic acid templates can comprise naturally-occurring, synthetic or recombinant nucleic acids. A nucleic acid template can comprise DNA, RNA or a DNA/RNA hybrid molecule. In some embodiments, a nucleic acid template can comprise any form of nucleic acid including chromosomal, genomic, organellar (e.g., mitochondrial, chloroplast or ribosomal), recombinant molecules, cloned, amplified (e.g., PCR amplified or emPCR), cDNA, RNA such as precursor mRNA or mRNA, oligonucleotide, or any type of nucleic acid library such as an amplicon library. In some embodiments, a nucleic acid template can be isolated from any source including from organisms such as prokaryotes, eukaryotes (e.g., humans, plants and animals), fungus, and viruses; cells; tissues; normal or diseased cells or tissues or organs, body fluids including blood, urine, serum, lymph, tumor, saliva, anal and vaginal secretions, amniotic samples, perspiration, and semen; environmental samples; culture samples; or synthesized nucleic acid molecules prepared using recombinant molecular biology or chemical synthesis methods. In some embodiments, a nucleic acid template can be chemically synthesized to include any type of nucleic acid analog. In some embodiments, a nucleic acid template can be isolated from a formalin-fixed tissue, or from a paraffin-embedded tissue, or from a formalin-fix paraffin-embedded (FFPE) tissue. In some embodiments, a nucleic acid template can be any length, including about 20-50 nucleotides, or about 50-100 nucleotides, or about 100-200 nucleotides, or about 200-300 nucleotides, or longer in length.

In some embodiments, the disclosure relates generally to methods (as well as related compositions, systems, and kits) for nucleotide polymerization, nucleotide incorporation, oligonucleotide synthesis, detecting nucleotide polymerization, detecting the presence of a nucleic acid, oligonucleotide amplification and detection of oligonucleotide amplification, wherein the method can be conducted with oligonucleotide probes (110). In some embodiments, an oligonucleotide probe comprises a first and second portion that can hybridize to a nucleic acid template, and a third portion that comprises little or no complementary sequences to form a single-stranded bubble structure. In some embodiments, an oligonucleotide probe can hybridize with a nucleic acid template where a first and second portion of the nucleic acid oligonucleotide probe forms a duplex with the nucleic acid template, and a third portion of the nucleic acid oligonucleotide probe forms a single-stranded bubble structure (FIGS. 1A and B). In some embodiments, the bubble structure resides between the first and second duplexes. In some embodiments, an oligonucleotide probe can hybridize with a nucleic acid template to form a transcription initiation bubble structure.

In some embodiments, an oligonucleotide probe can be designed to include sequences that are complementary to one or more nucleic acid template molecules. For example, a first and second portion of an oligonucleotide probe that hybridize to a nucleic acid template can be complementary to a region (or can be complementary to a region proximal to) a methylated region (e.g., CpG island), a tumor gene sequence, a tumor suppressor sequence, a single nucleotide polymorphism, a gene sequence of interest, or a genomic region of interest.

In some embodiments, an oligonucleotide probe comprises about 10-25 nucleotides, or about 25-50 nucleotides, or about 50-75 nucleotides, or about 75-100 nucleotides, or about 100-125 nucleotides, or about 125-150 nucleotides in length, or longer.

In some embodiments, a bubble structure includes about 5-15 nucleotides, or about 15-20 nucleotides, or about 20-25 nucleotides, or about 25-30 nucleotides, or about 30-35, or about 35-40 nucleotides, or about 40-45 nucleotides, or about 45-50 nucleotides in length or longer.

In some embodiments, an oligonucleotide probe comprises a natural or artificial promoter sequence. In some embodiments, an oligonucleotide probe comprises a promoter sequence that can be recognized by a polymerase. In some embodiments, an oligonucleotide probe comprises a sequence that can enhance the level of abortive initiation. In some embodiments, an oligonucleotide probe comprises a galP2 promoter sequence. In some embodiments, an oligonucleotide probe comprises a palindromic sequence.

In some embodiments, an oligonucleotide probe comprises a sequence and/or structure according to those disclosed in U.S. Pat. Nos. 7,045,319, 7,226,738, 7,468,261, 7,470,511, 7,473,775, 7,541,165, 8,211,644, 8,242,243, and 8,263,339 granted to Hanna, or published application U.S. 2010/0233709 by Hanna.

In some embodiments, an oligonucleotide probe (110) comprises DNA, RNA or DNA/RNA hybrid. In some embodiments, an oligonucleotide probe comprises a single-stranded nucleic acid. In some embodiments, an oligonucleotide probe comprises a nucleic acid isolated from natural source or a chemically synthesized nucleic acid.

In some embodiments, an oligonucleotide probe comprises a detectable reporter moiety joined to the 5′and/or 3′ end, or joined to any base, sugar, or phosphate group.

In some embodiments, the disclosure relates generally to methods (as well as related compositions, systems, and kits) for nucleotide polymerization, nucleotide incorporation, oligonucleotide synthesis, detecting nucleotide polymerization, detecting the presence of a nucleic acid, oligonucleotide amplification and detection of oligonucleotide amplification, wherein the method can be conducted with at least one macromolecule (120). In some embodiments, a macromolecule (120) can be attached or joined to an abortive initiation cassette. In some embodiments, a macromolecule (120) includes nucleic acids (e.g., DNA , RNA or DNA/RNA hybrids), polypeptides (e.g., proteins or enzymes), lipids, and sugars. In some embodiments, a macromolecule can be isolated from any source including from organisms such as prokaryotes, eukaryotes (e.g., humans, plants and animals), fungus, and viruses; cells; tissues; normal or diseased cells or tissues or organs, body fluids including blood, urine, serum, lymph, tumor, saliva, anal and vaginal secretions, amniotic samples, perspiration, and semen; environmental samples; culture samples; or synthesized nucleic acid molecules prepared using recombinant molecular biology or chemical synthesis methods. In some embodiments, a macromolecule can be isolated from a formalin-fixed tissue, or from a paraffin-embedded tissue, or from a formalin-fix paraffin-embedded (FFPE) tissue.

In some embodiments, the disclosure relates generally to methods (as well as related compositions, systems, and kits) for nucleotide polymerization, nucleotide incorporation, oligonucleotide synthesis, detecting nucleotide polymerization, detecting the presence of a nucleic acid, oligonucleotide amplification and detection of oligonucleotide amplification, wherein the method can be conducted with an abortive initiation cassette (130). In some embodiments, an abortive initiation cassette (130) comprises DNA, RNA or DNA/RNA hybrid. In some embodiments, an abortive initiation cassette comprises a single-stranded nucleic acid. In some embodiments an abortive initiation cassette comprises a nucleic acid isolated from natural source or a chemically synthesized nucleic acid. In some embodiments, an abortive initiation cassette comprises two or more single-stranded nucleic acids that interact with each other to form a single-stranded bubble region. In some embodiments, an abortive initiation cassette comprises one single-stranded nucleic acid comprising two or more portions having complementary sequences that can form intramolecular duplex regions. In some embodiments, an abortive initiation cassette comprises one or more portions having little or no complementary sequences that have little capacity to form intramolecular duplex regions. In some embodiments, an abortive initiation cassette can form duplex regions and a single-stranded bubble region (FIG. 2A). In some embodiments, an abortive initiation cassette can form a transcription initiation bubble structure.

In some embodiments, an abortive initiation cassette comprises about 10-25 nucleotides, or about 25-50 nucleotides, or about 50-75 nucleotides, or about 75-100 nucleotides, or about 100-125 nucleotides, or about 125-150 nucleotides in length, or about 150-200 nucleotides or longer.

In some embodiments, a bubble region includes about 5-15 nucleotides, or about 15-20 nucleotides, or about 20-25 nucleotides, or about 25-30 nucleotides, or about 30-35, or about 35-40 nucleotides, or about 40-45 nucleotides, or about 45-50 nucleotides in length or longer.

In some embodiments, an abortive initiation cassette can be designed to include sequences that are complementary to one or more nucleic acid template molecules. For example, a terminal 5′ or 3′ portion of an abortive initiation cassette can be complementary to a region (or can be complementary to a region proximal to) a methylated region (e.g., CpG island), a tumor gene sequence, a tumor suppressor sequence, a single nucleotide polymorphism, a gene sequence of interest, or a genomic region of interest.

In some embodiments, an abortive initiation cassette comprises a detectable reporter moiety joined to the 5′and/or 3′ end, or joined to any base, sugar, or phosphate group.

In some embodiments, an abortive initiation cassette comprises a natural or artificial promoter sequence. In some embodiments, an abortive initiation cassette comprises a promoter sequence that can be recognized by a polymerase. In some embodiments, an abortive initiation cassette comprises a sequence that can enhance the level of abortive initiation. In some embodiments, an abortive initiation cassette comprises a galP2 promoter sequence. In some embodiments, an abortive initiation cassette comprises a palindromic sequence.

In some embodiments, an abortive initiation cassette comprises a sequence and/or structure according to those disclosed in U.S. Pat. No. 7,045,319 to Hanna or published application U.S. 2010/0233709 to Hanna.

In some embodiments, the disclosure relates generally to methods (as well as related compositions, systems, and kits) for nucleotide polymerization, nucleotide incorporation, oligonucleotide synthesis, detecting nucleotide polymerization, detecting the presence of a nucleic acid, oligonucleotide amplification and detection of oligonucleotide amplification, wherein the method can be conducted with an initiator. In some embodiments, an initiator comprises a mononucleoside, mononucleotide or oligonucleotide (e.g., having two or more nucleosides), or analog thereof, which can serve as an RNA transcription primer. In some embodiments, an initiator comprises a nucleotide sequence that can be complementary to a portion of a transcription initiation bubble. In some embodiments, the transcription initiation bubble can be formed by hybridizing a nucleic acid template to an oligonucleotide probe, or can be formed by an abortive initiation cassette. In some embodiments, an initiator comprises a detectable reporter moiety or a binding partner.

In some embodiments, the disclosure relates generally to methods (as well as related compositions, systems, and kits) for nucleotide polymerization, nucleotide incorporation, oligonucleotide synthesis, detecting nucleotide polymerization, detecting the presence of a nucleic acid, oligonucleotide amplification and detection of oligonucleotide amplification, wherein the method can be practiced with an abortive initiation cassette which can be joined to a macromolecule (e.g., nucleic acid, protein, polypeptide, lipid or sugar) via a covalent or non-covalent bond, including an ionic bond, a hydrogen bond, an affinity bond, a dipole-dipole bond, a van der Waals bond, or a hydrophobic bond. In some embodiment, an abortive initiation cassette can be joined to a nucleic acid template molecule (or nucleic acid macromolecule) by hybridizing a portion of an abortive initiation cassette to a portion of a nucleic acid template molecule (FIGS. 2B and C). In some embodiments, an abortive initiation cassette can be joined to a macromolecule via a homobifunctional or a heterobifunctional cross-linking reagent. For example, a homobifunctional reagent can include two identical functional groups. A heterobifunctional reagent can include two dissimilar functional groups. For example, a heterobifunctional cross-linking agent can include a primary amine-reactive group and a thiol-reactive group. In some embodiments, a covalent cross-linking agent comprises a reagent capable of forming a disulfide (S—S), glycol (—CH(OH)—CH(OH)—), azo (—N═N—), sulfone (—S(═O₂—), ester (—C(═O)—O—), or amide (—C(═O)—N—) bridge. In some embodiments, a crosslinking agent comprises maleamides, iodoacetamides, and disulfies. Other examples of classes of crosslinking reagents include alpha-haloacetyl compounds, mercurials, aryl halides, acid anhydrides, anhydrides, isocyanates, isothiocyanates, sulfonyl halides, imidoesters, diazoacetates, diazonium salts, benzene-N₂—Cl⁻, and dicarbonyl compounds (S. S. Wong, in: “Chemistry of Protein Conjugation and Cross-Linking”, 1991, CRC Press, Inc., Boca Raton, USA). In some embodiments, an abortive initiation cassette can be joined to a macromolecule via interaction between binding partners attached to the abortive initiation cassette and macromolecule.

In some embodiments, the disclosure relates generally to methods (as well as related compositions, systems, and kits) for nucleotide polymerization, nucleotide incorporation, oligonucleotide synthesis, detecting nucleotide polymerization, detecting the presence of a nucleic acid, oligonucleotide amplification and detection of oligonucleotide amplification, wherein the method can be conducted with one or more nucleotides. For example, a nucleotide includes chain terminating nucleotides and elongation nucleotides. In some embodiments, synthesizing oligonucleotides can be conducted with at least one terminator nucleotide, at least one elongation nucleotide, or at least one terminator nucleotide and at least one elongation nucleotide. In some embodiments, a nucleotide comprises cytidine, thymidine, adenosine, guanosine, uridine or inosine. In some embodiments, a nucleotide comprises a detectable reporter moiety and/or one member of a binding partner (e.g., biotin).

In some embodiments, the disclosure relates generally to methods (as well as related compositions, systems, and kits) for nucleotide polymerization, nucleotide incorporation, oligonucleotide synthesis, detecting nucleotide polymerization, detecting the presence of a nucleic acid, oligonucleotide amplification and detection of oligonucleotide amplification, wherein the method can be conducted with one or more enzymes. For example, enzymes can catalyze nucleotide polymerization. In some embodiments, enzymes can comprise a polymerase. In some embodiments, enzymes can comprise a DNA-dependent RNA polymerases, DNA-dependent DNA polymerases, RNA-dependent RNA polymerases or RNA-dependent DNA polymerases. In some embodiments, enzymes can catalyze RNA transcription with or without a promoter sequence present on the nucleic acid template. In some embodiments, enzymes can catalyze synthesis of an oligonucleotide product having a sequence that can be complementary to a nucleic acid template. In some embodiments, enzymes can comprise an intact enzyme, or a biologically-active fragment thereof. In some embodiments, enzymes can comprise a single unit enzyme or multi-unit enzyme. In some embodiments, enzymes can comprise naturally-occurring polymerase, recombinant polymerase, mutant polymerase, variant polymerase, fusion or otherwise engineered polymerase, chemically modified polymerase, synthetic molecules, or analog, derivative or fragment thereof. In some embodiments, enzymes can comprise a prokaryotic, eukaryotic, viral or phage enzyme. In some embodiments, enzymes can comprise mesophilic or thermostable enzymes. In some embodiments, enzymes can polymerize nucleotides joined to a detectable reporter moiety or to a binding partner. In some embodiments, enzymes can comprise a wild-type or mutant enzyme. In some embodiments, mutant enzymes comprise any combination of insertions, deletions, and/or substitutions of one or more amino acids. In some embodiments, enzymes can be isolated from a cell, or generated using recombinant DNA technology or chemical synthesis methods. In some embodiments, enzymes can be post-translationally modified proteins or fragments thereof. In some embodiments, enzymes can comprise two or more portions of polymerases linked together. In some embodiments, enzymes can comprise a fusion protein comprising at least two portions linked to each other, where the first portion comprises a polypeptide that can catalyze nucleotide polymerization and a second portion comprising a second polypeptide. In some embodiments, enzymes can comprise other enzymatic activities, such as for example, 3′ to 5′ or 5′ to 3′ exonuclease activity. In some embodiments, an enzyme comprises an RNA polymerase from: Escherichia coli, Escherichia coli bacteriophage T7, Escherichia coli bacteriophage T3, Salmonella typhimurium bacteriophage SP6; RNA-dependent RNA polymerases, including poliovirus RNA polymerase; reverse transcriptases including HIV reverse transcriptase; and DNA polymerases including Escherichia coli, T7, T4 DNA polymerase, Taq thermostable DNA polymerase, terminal transferase, primase, and telomerase.

In some embodiments, the disclosure relates generally to methods (as well as related compositions, systems, and kits) for nucleotide polymerization, nucleotide incorporation, oligonucleotide synthesis, detecting nucleotide polymerization, detecting the presence of a nucleic acid, oligonucleotide amplification and detection of oligonucleotide amplification, wherein the method can be conducted with or on a support. In some embodiments, a support can be contacted with any component of a method for synthesizing oligonucleotides, including a nucleic acid template, target macromolecule, oligonucleotide probe and abortive initiation cassette. For example, a nucleic acid template can be hybridized to an oligonucleotide probe to form a transcription initiation bubble structure, and the nucleic acid template and/or the oligonucleotide probe can be attached to a support. Optionally, a macromolecule can be attached to an abortive initiation cassette (AIC) and the macromolecule and/or the abortive initiation cassette can be attached to a support.

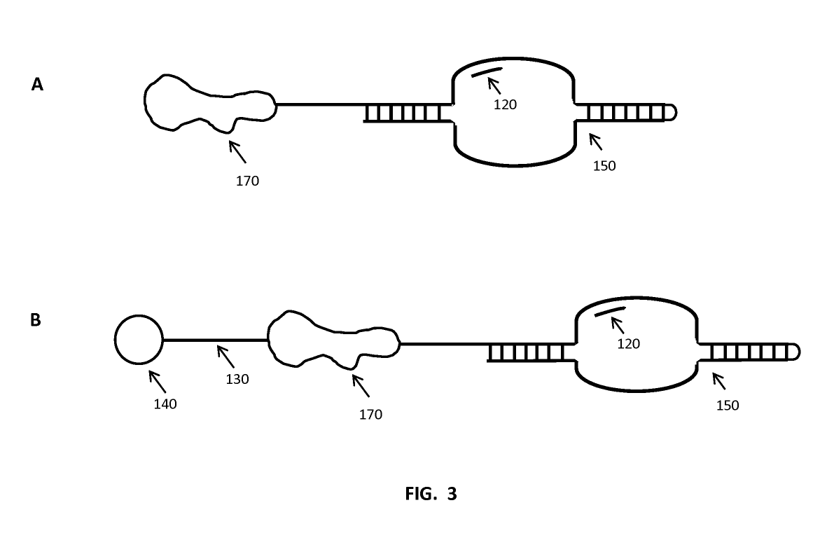

In some embodiments, a linker molecule can attach a nucleic acid template, target macromolecule, oligonucleotide probe or abortive initiation cassette to a support. In some embodiments, a linker molecule can attach to: a 5′ or 3′ end of a nucleic acid template; a 5′ or 3′ end of an oligonucleotide probe; a 5′ or 3′ end of a nucleic acid macromolecule; a 5′ or 3′ end of an abortive initiation cassette, or; a terminal-amino end or a terminal-carboxyl end or an internal portion of a polypeptide. In some embodiments, a linker molecule can attach to any portion of a lipid or sugar. In some embodiments, a linker molecule can be a capture oligonucleotide. In some embodiments, a 5′ or 3′ end of a capture oligonucleotide can be attached to a support. In some embodiments, a linker molecule comprises an antibody or binding partner. For example, a nucleic acid template can be hybridized to an oligonucleotide probe to form a transcription initiation bubble structure, and the nucleic acid template and/or the oligonucleotide probe can be attached to a linker molecule which can be attached to a support (FIG. 1B). A macromolecule can be joined to an abortive initiation cassette (AIC), and the macromolecule and/or the abortive initiation cassette can be attached to a to a linker molecule (e.g., antibody or binding partner) (130) which can be attached to a support (FIGS. 2C and 3B).

In some embodiments, a support can have a surface. In some embodiments, a surface can be an outer or top-most layer or boundary of an object. In some embodiments, a surface can be located in the interior of an object, including for example, an internal three-dimensional scaffold. In some embodiments, a support can include a solid surface or semi-solid surface. In some embodiments, a surface can be porous, semi-porous or non-porous. In some embodiments, a support can have a planar surface, as well as concave, convex, or any combination thereof. In some embodiments, a support can be a bead, particle, sphere, filter, flowcell, or gel. In some embodiments, a support includes the inner walls of a capillary, a channel, a well, groove, channel, reservoir. In some embodiments, a support can include texture (e.g., etched, cavitated, pores, three-dimensional scaffolds or bumps).

In some embodiments, a support comprises a particle having a shape that is spherical, hemispherical, cylindrical, barrel-shaped, toroidal, rod-like, disc-like, conical, triangular, cubical, polygonal, tubular, wire-like or irregular. A particle can have an iron core or comprise a hydrogel or agarose (e.g., Sepharose™). A particle can be paramagnetic. A particle can be spherical or irregular shape. A particle can have cavitation or pores, or can include three-dimensional scaffolds.

In some embodiments, a support can comprise an inorganic material, natural polymers, synthetic polymers, or non-polymeric material. In some embodiments, a support can be made from materials such as glass, borosilicate glass, silica, quartz, fused quartz, mica, polyacrylamide, plastic polystyrene, polycarbonate, polymethacrylate (PMA), polymethyl methacrylate (PMMA), polydimethylsiloxane (PDMS), silicon, germanium, graphite, ceramics, silicon, semiconductor, high refractive index dielectrics, crystals, gels, polymers, or films (e.g., films of gold, silver, aluminum, or diamond). In some embodiments, nucleic acid fragments can be arranged on a support in a random pattern, organized pattern, rectilinear pattern, hexagonal pattern, or addressable array pattern. In some embodiments, a support can be coated with an acrylamide compound.

In some embodiments, an oligonucleotide synthesis reaction can be attached to a support by binding a chemical compound on the support with another chemical compound on a nucleic acid template, target macromolecule, oligonucleotide probe or abortive initiation cassette. For example, a 5′ or 3′ end of a nucleic acid molecule can be modified to include an amino group that can bind to a carboxylic acid compound or amine on a support. In some embodiments, 5′ end can include a phosphate group for reacting with an amine-coated support in the presence of a carbodiimide (e.g., water soluble carbodiimide). In some embodiments, a nucleic acid can be biotinylated at one end to bind with an avidin-like compound (e.g. streptavidin) attached to a support. In some embodiments, one end of a nucleic acid molecule can include hybridize with a capture adaptor/primer sequence which is attached to a support. In some embodiments, an Ion Sphere™ Particle (sold as a component of the Ion Xpress Template Kit (Part No. 4469001)) can be attached to a nucleic acid template, target macromolecule, oligonucleotide probe or abortive initiation cassette for amplification. Immobilizing nucleic acids to an Ion Sphere™ Particle can be performed essentially according to the protocols provided in the Ion Xpress™ Template Kit v2.0 User Guide (Part No.: 4469004)).

In some embodiments, a plurality of particles can be deposited to a surface of a sequencing instrument or onto a sensor that that senses the presence of byproducts from a nucleotide polymerization or from a nucleotide incorporation reaction. In some embodiments, byproducts include pyrophosphate, hydrogen ion, charge transfer, and heat. In some embodiments, a sensor can be a field-effect transistor (FET). Sequencing reagents can be delivered to the deposited particles to conduct sequencing reactions. In some embodiments, a nucleic acid template or abortive initiation cassette can be attached or immobilized to Ion Sphere™ Particles (sold as a component of the Ion Xpress Template Kit (Part No. 4469001)) for clonal amplification and used to conduct oligonucleotide synthesis or sequencing reactions. Immobilization to Ion Sphere™ Particles can be performed essentially according to the protocols provided in the Ion Xpress™ Template Kit v2.0 User Guide (Part No.: 4469004)).

In some embodiments, the disclosure relates generally to methods (as well as related compositions, systems, and kits) for nucleotide polymerization, nucleotide incorporation, oligonucleotide synthesis, detecting nucleotide polymerization, detecting the presence of a nucleic acid, oligonucleotide amplification and detection of oligonucleotide amplification, wherein the method can be conducted on a support having a surface that is in contact with or capacitively coupled to a sensor that can detect one or more byproducts of nucleotide incorporation or nucleotide incorporation. In some embodiments, the byproducts include pyrophosphate, hydrogen ion, charge transfer, and heat.

In some embodiments, a nucleotide incorporation reaction comprises an abortive transcription initiation reaction to reiteratively synthesis oligonucleotides. In some embodiments, an abortive transcription initiation reaction can be conducted on or near an ion-sensitive sensor. In some embodiments, a sensor can be a field-effect transistor (FET). For example, a sensor can be an ion-sensitive sensor used for nucleic acid sequencing.

In some embodiments, detection of nucleotide incorporation by detecting physicochemical byproducts of the extension reaction, can include pyrophosphate, hydrogen ion, charge transfer, heat, and the like, as disclosed, for example, in Pourmand et al, Proc. Natl. Acad. Sci., 103: 6466-6470 (2006); Purushothaman et al., IEEE ISCAS, IV-169-172; Rothberg et al, U.S. Patent Publication No. 2009/0026082; Anderson et al, Sensors and Actuators B Chem., 129: 79-86 (2008); Sakata et al., Angew. Chem. 118:2283-2286 (2006); Esfandyapour et al., U.S. Patent Publication No. 2008/01666727; and Sakurai et al., Anal. Chem. 64: 1996-1997 (1992).

Reactions involving the generation and detection of ions are widely performed. The use of direct ion detection methods to monitor the progress of such reactions can simplify many current biological assays. For example, template-dependent nucleic acid synthesis by a polymerase can be monitored by detecting hydrogen ions that are generated as natural byproducts of nucleotide incorporations catalyzed by the polymerase. Ion-sensitive detection (also referred to as “pH-based” or “ion-based” detection) exploits the direct detection of ionic byproducts, such as hydrogen ions, that are produced as a byproduct of nucleotide incorporation. In one exemplary system for ion-based detection, the nucleic acid undergoing abortive transcription initiation can be captured in a microwell, and nucleotides can be floated across the well, one at a time, under nucleotide incorporation conditions. The polymerase incorporates the appropriate nucleotide into the growing strand, and the hydrogen ion that is released can change the pH in the solution, which can be detected by an ion sensor. This technique does not require labeling of the nucleotides or expensive optical components, and allows for far more rapid completion of nucleotide polymerization runs. Examples of such ion-based nucleotide polymerization methods and platforms include the Ion Torrent PGM™ sequencer (Ion Torrent™ Systems, Life Technologies Corporation).

In some embodiments, one or more abortive transcription initiation systems produced using the methods, compositions and kits of the present teachings can be used as a substrate for a biological or chemical reaction that is detected and/or monitored by a sensor including a field-effect transistor (FET). In various embodiments the FET is a chemFET or an ISFET. A “chemFET” or chemical field-effect transistor, is a type of field effect transistor that acts as a chemical sensor. It is the structural analog of a MOSFET transistor, where the charge on the gate electrode is applied by a chemical process. An “ISFET” or ion-sensitive field-effect transistor, is used for measuring ion concentrations in solution; when the ion concentration (such as H+) changes, the current through the transistor will change accordingly. A detailed theory of operation of an ISFET is given in “Thirty years of ISFETOLOGY: what happened in the past 30 years and what may happen in the next 30 years,” P. Bergveld, Sens. Actuators, 88 (2003), pp. 1-20.

In some embodiments, the FET may be a FET array. As used herein, an “array” is a planar arrangement of elements such as sensors or wells. The array may be one or two dimensional. A one dimensional array can be an array having one column (or row) of elements in the first dimension and a plurality of columns (or rows) in the second dimension. The number of columns (or rows) in the first and second dimensions may or may not be the same. The FET or array can comprise 102, 103, 104, 105, 106, 107 or more FETs.

In some embodiments, one or more microfluidic structures can be fabricated above the FET sensor array to provide for containment and/or confinement of a biological or chemical reaction. For example, in one implementation, the microfluidic structure(s) can be configured as one or more wells (or microwells, or reaction chambers, or reaction wells, as the terms are used interchangeably herein) disposed above one or more sensors of the array, such that the one or more sensors over which a given well is disposed detect and measure analyte presence, level, and/or concentration in the given well. In some embodiments, there can be a 1:1 correspondence of FET sensors and reaction wells.

Microwells or reaction chambers are typically hollows or wells having well-defined shapes and volumes which can be manufactured into a substrate and can be fabricated using conventional microfabrication techniques, e.g. as disclosed in the following references: Doering and Nishi, Editors, Handbook of Semiconductor Manufacturing Technology, Second Edition (CRC Press, 2007); Saliterman, Fundamentals of BioMEMS and Medical Microdevices (SPIE Publications, 2006); Elwenspoek et al, Silicon Micromachining (Cambridge University Press, 2004); and the like. Examples of configurations (e.g. spacing, shape and volumes) of microwells or reaction chambers are disclosed in Rothberg et al, U.S. patent publication 2009/0127589; Rothberg et al, U.K. patent application GB24611127.

In some embodiments, the biological or chemical reaction can be performed in a solution or a reaction chamber that is in contact with or capacitively coupled to a FET such as a chemFET or an ISFET. The FET (or chemFET or ISFET) and/or reaction chamber can be an array of FETs or reaction chambers, respectively.

In some embodiments, a biological or chemical reaction can be carried out in a two-dimensional array of reaction chambers, wherein each reaction chamber can be coupled to a FET, and each reaction chamber is no greater than 10 μm³ (i.e., 1 pL) in volume. In some embodiments each reaction chamber is no greater than 0.34 pL, 0.096 pL or even 0.012 pL in volume. A reaction chamber can optionally be 22, 32, 42, 52, 62, 72, 82, 92, or 102 square microns in cross-sectional area at the top. Preferably, the array has at least 102, 103, 104, 105, 106, 107,108, 109, or more reaction chambers. In some embodiments, the reaction chambers can be capacitively coupled to the FETs.

FET arrays as used in various embodiments according to the disclosure can be fabricated according to conventional CMOS fabrications techniques, as well as modified CMOS fabrication techniques and other semiconductor fabrication techniques beyond those conventionally employed in CMOS fabrication. Additionally, various lithography techniques can be employed as part of an array fabrication process.

Exemplary FET arrays suitable for use in the disclosed methods, as well as microwells and attendant fluidics, and methods for manufacturing them, are disclosed, for example, in U.S. Patent Publication No. 20100301398; U.S. Patent Publication No. 20100300895; U.S. Patent Publication No. 20100300559; U.S. Patent Publication No. 20100197507, U.S. Patent Publication No. 20100137143; U.S. Patent Publication No. 20090127589; and U.S. Patent Publication No. 20090026082, which are incorporated by reference in their entireties.

In one aspect, the disclosed methods, compositions, systems, apparatuses and kits can be used for carrying out label-free nucleotide polymerization, and in particular, ion-based detection of nucleotide polymerization. The concept of label-free detection of nucleotide incorporation has been described in the literature, including the following references that are incorporated by reference: Rothberg et al, U.S. patent publication 2009/0026082; Anderson et al, Sensors and Actuators B Chem., 129: 79-86 (2008); and Pourmand et al, Proc. Natl. Acad. Sci., 103: 6466-6470 (2006). Briefly, in nucleic acid sequencing applications, nucleotide incorporations are determined by measuring natural byproducts of polymerase-catalyzed extension reactions, including hydrogen ions, polyphosphates, PPi, and Pi (e.g., in the presence of pyrophosphatase). Examples of such ion-based nucleic acid sequencing methods and platforms include the Ion Torrent PGM™ sequencer (Ion Torrent™ Systems, Life Technologies Corporation).

In some embodiments, the disclosure relates generally to detecting nucleotide polymerization (or determining a nucleotide sequence) using the abortive transcription initiation methods, compositions, systems and kits provided by the teachings herein. In some embodiments, methods for detecting abortive transcription initiation can be performed near or on an ion-sensitive sensor. In some embodiments, methods for detecting abortive transcription initiation comprise: conducting an abortive transcription initiation reaction on a sensor that senses the presence of byproducts from a nucleotide polymerization reaction, wherein the abortive transcription initiation reaction includes a nucleic acid template hybridized to an oligonucleotide probe to form a transcription initiation bubble structure, an initiator, one or more nucleotides, and at least one polymerase, so as to polymerize the one or more nucleotides onto the initiator; and detecting the nucleotide polymerization by a change in an electrical parameter at the FET. In some embodiments, methods for detecting abortive transcription initiation comprise: conducting an abortive transcription initiation reaction on a sensor that senses the presence of byproducts from a nucleotide polymerization reaction, wherein the abortive transcription initiation reaction includes a nucleic acid template attached to an abortive initiation cassette that forms a transcription initiation bubble structure, an initiator, one or more nucleotides, and at least one polymerase, so as to polymerize the one or more nucleotides onto the initiator; and detecting the nucleotide polymerization by a change in an electrical parameter at the sensor. In some embodiments, the initiator comprises a mononucleoside, a mononucleotide or an oligonucleotide having two or more nucleosides. In some embodiments, the initiator comprises a nucleotide sequence that can be complementary to a portion of a transcription initiation bubble. In some embodiments, the one or more nucleotides comprise chain terminating nucleotides or elongation nucleotides. In some embodiments, the polymerase comprises a DNA-dependent RNA polymerases, DNA-dependent DNA polymerases, RNA-dependent RNA polymerases or RNA-dependent DNA polymerases. In some embodiments, the nucleic acid template or the oligonucleotide probe can be attached to a surface via a linker molecule. In some embodiments, the nucleic acid template or the abortive initiation cassette can be attached to a surface via a linker molecule. In some embodiments, the surface comprises a planar surface or a particle. In some embodiments, the nucleic acid template or the oligonucleotide probe attached to the particle can be deposited onto a sensor that senses the presence of byproducts from a nucleotide polymerization reaction. In some embodiments, the byproducts from a nucleotide polymerization reaction comprise pyrophosphate, hydrogen ion, charge transfer or heat. In some embodiments, the sensor comprises a field-effect transistor (FET). In some embodiments, polymerizing one or more nucleotides onto the initiator produces byproducts comprising pyrophosphate, hydrogen ion, charge transfer or heat. In some embodiments, the sensor detects production of a byproduct thereby detecting abortive transcription initiation.

In some embodiments, the template-dependent synthesis includes incorporating one or more nucleotides in a template-dependent fashion into a newly synthesized nucleic acid strand.

Optionally, the methods can further include producing one or more ionic byproducts of such nucleotide incorporation.

In some embodiments, the methods can further include detecting the incorporation of the one or more nucleotides into the initiator. Optionally, the detecting can include detecting the release of hydrogen ions.

In another embodiment, the disclosure relates generally to a method for detecting abortive transcription initiation, comprising: (a) producing a plurality of abortive transcription initiation system according to the methods disclosed herein; (b) disposing a plurality of abortive transcription initiation systems into a plurality of reaction chambers, wherein one or more of the reaction chambers are in contact with a field effect transistor (FET). Optionally, the method further includes contacting at least one of the abortive transcription initiation system disposed into one of the reaction chambers with a polymerase, thereby synthesizing a new nucleic acid strand by sequentially incorporating one or more nucleotides into a nucleic acid molecule. Optionally, the method further includes generating one or more hydrogen ions as a byproduct of such nucleotide incorporation. Optionally, the method further includes detecting the incorporation of the one or more nucleotides by detecting the generation of the one or more hydrogen ions using the FET.

In some embodiments, the detecting includes detecting a change in voltage and/or current at the at least one FET within the array in response to the generation of the one or more hydrogen ions.

In some embodiments, the FET can be selected from the group consisting of: ion-sensitive FET (isFET) and chemically-sensitive FET (chemFET).

One exemplary system involving detecting ionic byproducts of nucleotide incorporation is the Ion Torrent PGM™ sequencer (Life Technologies), which is an ion-based sequencing system that sequences nucleic acid templates by detecting hydrogen ions produced as a byproduct of nucleotide incorporation. Typically, hydrogen ions are released as byproducts of nucleotide incorporations occurring during template-dependent nucleic acid synthesis by a polymerase. The Ion Torrent PGM™ sequencer detects the nucleotide incorporations by detecting the hydrogen ion byproducts of the nucleotide incorporations. The Ion Torrent PGM™ sequencer can include a plurality of nucleic acid templates to be sequenced, each template disposed within a respective sequencing reaction well in an array. The wells of the array can each be coupled to at least one ion sensor that can detect the release of H⁺ ions or changes in solution pH produced as a byproduct of nucleotide incorporation. The ion sensor comprises a field effect transistor (FET) coupled to an ion-sensitive detection layer that can sense the presence of H⁺ ions or changes in solution pH. The ion sensor can provide output signals indicative of nucleotide incorporation which can be represented as voltage changes whose magnitude correlates with the H⁺ ion concentration in a respective well or reaction chamber. Different nucleotide types can be flowed serially into the reaction chamber, and can be incorporated by the polymerase into an extending primer (or polymerization site) in an order determined by the sequence of the template. Each nucleotide incorporation can be accompanied by the release of H⁺ ions in the reaction well, along with a concomitant change in the localized pH. The release of H⁺ ions can be registered by the FET of the sensor, which produces signals indicating the occurrence of the nucleotide incorporation. Nucleotides that are not incorporated during a particular nucleotide flow may not produce signals. The amplitude of the signals from the FET can also be correlated with the number of nucleotides of a particular type incorporated into the extending nucleic acid molecule thereby permitting homopolymer regions to be resolved. Thus, during a run of the sequencer multiple nucleotide flows into the reaction chamber along with incorporation monitoring across a multiplicity of wells or reaction chambers can permit the instrument to resolve the sequence of many nucleic acid templates simultaneously. Further details regarding the compositions, design and operation of the Ion Torrent PGM™ sequencer can be found, for example, in U.S. patent application Ser. No. 12/002,781, now published as U.S. Patent Publication No. 2009/0026082; U.S. Patent application Ser. No. 12/474,897, now published as U.S. Patent Publication No. 2010/0137143; and U.S. patent application Ser. No. 12/492,844, now published as U.S. Patent Publication No. 2010/0282617, all of which applications are incorporated by reference herein in their entireties.

In some embodiments, the disclosure relates generally to use of abortive transcription initiation systems produced using any of the methods, compositions and kits of the present disclosure in methods of ion-based sequencing.

In a typical embodiment of ion-based nucleic acid sequencing, nucleotide incorporations can be detected by detecting the presence and/or concentration of hydrogen ions generated by polymerase-catalyzed extension reactions. In one embodiment, templates each having a primer and polymerase operably bound can be loaded into reaction chambers (such as the microwells disclosed in Rothberg et al, cited herein), after which repeated cycles of nucleotide addition and washing can be carried out. In some embodiments, such templates can be attached as clonal populations to a solid support, such as particles, bead, or the like, and said clonal populations are loaded into reaction chambers. As used herein, “operably bound” means that a primer is annealed to a template so that the primer's 3′ end may be extended by a polymerase and that a polymerase is bound to such primer-template duplex, or in close proximity thereof so that binding and/or extension takes place whenever nucleotides are added.

In each addition step of the cycle, the polymerase can extend the primer by incorporating added nucleotide only if the next base in the template is the complement of the added nucleotide. If there is one complementary base, there is one incorporation, if two, there are two incorporations, if three, there are three incorporations, and so on. With each such incorporation there is a hydrogen ion released, and collectively a population of templates releasing hydrogen ions changes the local pH of the reaction chamber. The production of hydrogen ions is monotonically related to the number of contiguous complementary bases in the template (as well as the total number of template molecules with primer and polymerase that participate in an extension reaction). Thus, when there are a number of contiguous identical complementary bases in the template (i.e. a homopolymer region), the number of hydrogen ions generated, and therefore the magnitude of the local pH change, can be proportional to the number of contiguous identical complementary bases. If the next base in the template is not complementary to the added nucleotide, then no incorporation occurs and no hydrogen ion is released. In some embodiments, after each step of adding a nucleotide, an additional step can be performed, in which an unbuffered wash solution at a predetermined pH is used to remove the nucleotide of the previous step in order to prevent misincorporations in later cycles. In some embodiments, the after each step of adding a nucleotide, an additional step can be performed wherein the reaction chambers are treated with a nucleotide-destroying agent, such as apyrase, to eliminate any residual nucleotides remaining in the chamber, which may result in spurious extensions in subsequent cycles.

In one exemplary embodiment, different kinds of nucleotides are added sequentially to the reaction chambers, so that each reaction can be exposed to the different nucleotides one at a time. For example, nucleotides can be added in the following sequence: dATP, dCTP, dGTP, dTTP, dATP, dCTP, dGTP, dTTP, and so on; with each exposure followed by a wash step. The cycles may be repeated for 50 times, 100 times, 200 times, 300 times, 400 times, 500 times, 750 times, or more, depending on the length of sequence information desired.

In some embodiments, sequencing can be performed according to the user protocols supplied with the PGM™ sequencer. Example 3 provides one exemplary protocol for ion-based sequencing using the Ion Torrent PGM™ sequencer (Ion Torrent™ Systems, Life Technologies, CA).

In some embodiments, the disclosure relates generally to systems (as well as related compositions, methods, and kits) for nucleotide polymerization, oligonucleotide synthesis, detecting nucleotide polymerization, detecting the presence of a nucleic acid, oligonucleotide amplification and detection of oligonucleotide amplification, wherein the systems comprise any one or any combination of: a transcription initiation bubble, nucleic acid template, an oligonucleotide probe, a macromolecule, an abortive initiation cassette, one or more initiators, at least one enzyme, one or more nucleotides (e.g., chain terminating nucleotides and/or elongation nucleotides), a linker molecule, a surface and/or a sensor that senses the presence of byproducts from a nucleotide polymerization reaction.

In some embodiments, a system comprises any combination of a nucleic acid template, an oligonucleotide probe, one or more initiators, at least one enzyme, one or more nucleotides, and/or a sensor that senses the presence of byproducts from a nucleotide polymerization reaction. In some embodiments, a nucleic acid template can hybridize to an oligonucleotide probe to form a transcription initiation bubble structure. In some embodiments, an oligonucleotide probe can hybridize with a nucleic acid template to form a transcription initiation bubble structure. In some embodiments, an initiator comprises a mononucleoside, mononucleotide or oligonucleotide (e.g., having two or more nucleosides), or analog thereof. In some embodiments, an initiator comprises a nucleotide sequence that can be complementary to a portion of a transcription initiation bubble. In some embodiments, one or more nucleotides comprise chain terminating nucleotides and/or elongation nucleotide. In some embodiments, an enzyme can be a polymerase, terminal transferase, primase, or telomerase. In some embodiments, an enzyme can be a DNA-dependent RNA polymerases, DNA-dependent DNA polymerases, RNA-dependent RNA polymerases or RNA-dependent DNA polymerases. In some embodiments, a nucleic acid template and/or an oligonucleotide probe can be attached to a surface via a linker molecule. In some embodiments, a surface can be a planar surface or a particle. In some embodiments, a particle (attached to nucleic acid template and/or an oligonucleotide probe) can be deposited onto a sensor that senses the presence of byproducts from a nucleotide polymerization reaction. In some embodiments, a byproduct from a nucleotide polymerization reaction includes pyrophosphate, hydrogen ion, charge transfer, and heat. In some embodiments, a sensor can be a field-effect transistor (FET).

In some embodiments, a system comprises any combination of a macromolecule, an abortive initiation cassette, one or more initiators, at least one enzyme, one or more nucleotides (e.g., chain terminating nucleotides and/or elongation nucleotides), a linker molecule, a surface and/or a sensor that senses the presence of byproducts from a nucleotide polymerization reaction. In some embodiments, macromolecules include nucleic acids, polypeptides, lipids and sugars. In some embodiments, an abortive initiation cassette comprises a single-stranded nucleic acid. In some embodiments, an abortive initiation cassette can form duplex regions and a single-stranded bubble region. In some embodiments, a macromolecule can be attached to an abortive initiation cassette. In some embodiments, an initiator comprises a mononucleoside, mononucleotide or oligonucleotide (e.g., having two or more nucleosides), or analog thereof. In some embodiments, an initiator comprises a nucleotide sequence that can be complementary to a portion of a transcription initiation bubble. In some embodiments, one or more nucleotides comprise chain terminating nucleotides and/or elongation nucleotide. In some embodiments, an enzyme can be a polymerase, terminal transferase, primase, or telomerase. In some embodiments, an enzyme can be a DNA-dependent RNA polymerases, DNA-dependent DNA polymerases, RNA-dependent RNA polymerases or RNA-dependent DNA polymerases. In some embodiments, a macromolecule and/or an abortive initiation cassette can be attached to a surface via a linker molecule. In some embodiments, a surface can be a planar surface or a particle. In some embodiments, a particle (attached to a macromolecule and/or an abortive initiation cassette) can be deposited onto a sensor that senses the presence of byproducts from a nucleotide polymerization reaction. In some embodiments, a byproduct from a nucleotide polymerization reaction includes pyrophosphate, hydrogen ion, charge transfer, and heat. In some embodiments, a sensor can be a field-effect transistor (FET).

In some embodiments, the disclosure relates generally to kits (as well as related compositions, systems, and methods) for nucleotide polymerization, oligonucleotide synthesis, detecting nucleotide polymerization, detecting the presence of a nucleic acid, oligonucleotide amplification and detection of oligonucleotide amplification, wherein the kits comprise any one or any combination of reagents and/or components for synthesizing oligonucleotides. In some embodiments, a kit can include any combination of: a transcription initiation bubble, a nucleic acid template (e.g., a test or control template), an oligonucleotide probe, a macromolecule (e.g., a test or control macromolecule), an abortive initiation cassette, one or more initiators, at least one enzyme, one or more nucleotides (e.g., chain terminating nucleotides and/or elongation nucleotides), a linker molecule, a surface (e.g., planar surface or particles), and/or a sensor that senses the presence of byproducts from a nucleotide polymerization reaction. In some embodiments, kits can include buffers and reagents for hybridizing a nucleic acid template to an oligonucleotide probe to form a transcription initiation bubble structure, or for forming a transcription initiation bubble structure with an abortive initiation cassette. In some embodiments, kits can include buffers and reagents for conducting an abortive transcription initiation reaction. In some embodiments, kits can include a set of instructions in print or in digital form.

While the principles of the present teachings have been described in connection with specific embodiments of reiterative oligonucleotide synthesis, it should be understood clearly that these descriptions are made only by way of example and are not intended to limit the scope of the present teachings or claims. What has been disclosed herein has been provided for the purposes of illustration and description. It is not intended to be exhaustive or to limit what is disclosed to the precise forms described. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated. It is intended that the scope of what is disclosed be defined by the following claims and their equivalents.

What is claimed:

1. An oligonucleotide synthesis system comprising:
(a) at least one surface capacitively coupled to one or more sensors that detect a nucleotide polymerization reaction byproduct; and
(b) an abortive transcription initiation reaction.

2. The system of claim 1, wherein the one or more sensors are configured to detect the presence of a nucleotide polymerization reaction byproduct at the surface.

3. The system of claim 1, wherein the one or more sensors comprise a field-effect transistor (FET).

4. The system of claim 1, wherein the one or more sensors comprise a field-effect transistor (FET), ion-sensitive field-effect transistors (ISFET), chemical-sensitive field-effect transistors (chemFET), or biologically active field-effect transistors (bioFET).

5. The system of claim 1, wherein the abortive transcription initiation reaction synthesizes oligonucleotide.

6. The system of claim 1, wherein the abortive transcription initiation reaction generates nucleotide incorporation byproducts.

7. The system of claim 1, wherein the nucleotide polymerization reaction byproducts comprise a pyrophosphate, a hydrogen ion, charge transfer or heat.

8. The system of claim 1, wherein the abortive transcription initiation reaction comprises a transcription initiation bubble and optionally comprises any one or any combination of (i) a transcription initiator, (ii) one or more nucleotides, (iii) and/or at least one polymerase.

9. A method for synthesizing oligonucleotides, comprising: generating a change in an electrical parameter of a sensor by conducting an abortive transcription initiation reaction on at least one surface that is capacitively coupled to one or more sensors that detect a nucleotide polymerization reaction byproduct.

10. The method of claim 9, wherein the at least one surface comprises a plurality of surfaces.

11. The method of claim 10, wherein the plurality of surfaces comprises an array of two or more surfaces.

12. The method of claim 9, wherein the one or more sensors are configured to detect the presence of a nucleotide polymerization reaction byproduct at the surface.

13. The method of claim 9, wherein the one or more sensors comprise a field-effect transistor (FET).

14. The method of claim 9, wherein the one or more sensors comprise a field-effect transistor (FET), ion-sensitive field-effect transistors (ISFET), chemical-sensitive field-effect transistors (chemFET), or biologically active field-effect transistors (bioFET).

15. The method of claim 9, wherein the abortive transcription initiation reaction synthesizes oligonucleotide.

16. The method of claim 9, wherein the abortive transcription initiation reaction generates nucleotide incorporation byproducts.

17. The method of claim 9, wherein the nucleotide polymerization reaction byproduct comprises a pyrophosphate, a hydrogen ion, charge transfer or heat.

18. The method of claim 9, wherein the abortive transcription initiation reaction comprises a transcription initiation bubble and optionally comprises any one or any combination of (i) a transcription initiator, (ii) one or more nucleotides, and/or (iii) at least one polymerase.

19. The method of claim 18, wherein the transcription initiation bubble comprises two nucleic acid strands hybridized together to form a structure having first and second duplex regions flanking a bubble region.

20. The method of claim 18, wherein the transcription initiation bubble comprises a single nucleic acid strand having intramolecular hybridization regions to form a structure having first and second duplex regions flanking a bubble region.
